(19) United States
(12) Patent Application Publication
(10) Pub. No.: US 2002/0187252 A1
(43) Pub. Date: Dec. 12, 2002

(54) High superabsorbent content webs and a method for making them

(76) Inventors: Jaspreet Singh, Montreal (CA); Richard N. Dodge, Appleton, WI (US)

Correspondence Address: Kimberly J. Prior, Esq., KILPATRICK STOCKTON LLP, 2400 Monarch Tower, 3424 Peachtree Road, N.E., Atlanta, GA, 30326, US

(21) Appl. No.: 09/815,159

(22) Filed: Mar. 22, 2001

(51) Int. Cl. B05D 3/00; A61F 13/15; B05D 1/36; A61F 13/20

(52) U.S. Cl. 427/2.31; 604/367; 427/202

(57) ABSTRACT

The present invention is directed to webs comprising fibers and superabsorbent materials in which the webs exhibit low susceptibility to migration of superabsorbent material within the web, even where the webs contain very high concentrations of superabsorbent materials. The invention is further directed to absorbent articles comprising such webs. The invention is further directed to methods for manufacturing such webs.

### FIELD OF THE INVENTION

The present invention is directed to webs comprising fibers and superabsorbent materials that have very low susceptibility to migration of superabsorbent material within the webs, even when the webs contain very high concentrations of superabsorbent materials. The invention is further directed to methods for manufacturing such webs. The present invention is further directed to absorbent articles containing the webs of the present invention.

### BACKGROUND OF THE INVENTION

Superabsorbent materials (referred to herein as SAM or SAMs) are hydrogel-forming polymeric materials widely used in disposable absorbent products such as diapers, training pants, adult incontinence products, and feminine care products. Although the structure of a disposable absorbent product has many components, in many instances the in-use performance of the product is directly related to the amount and characteristics of the absorbent material contained within the product. SAMs are commonly included in absorbent materials because they can absorb liquids in amounts many times their mass. Use of SAMs in the absorbent component of such products has therefore improved the absorbency of the products and has reduced their tendency to leak. Absorbent materials also commonly include fibrous materials along with the SAM. The addition of fibrous material to the absorbent component reduces the potential for leakage of an absorbent component by temporarily holding the liquid until the SAM absorbs it. Fibers also serve to separate the particles of SAM to avoid situations in which particles of SAM deform during swelling and block the interstitial spaces between the particles, or between the particles and the fibers, thus preventing the flow of liquid through the interstitial spaces.

A common way of combining SAM and fibers to form absorbent material is to mix particles of SAM with fibers and to form webs from the resulting mixture. Webs that contain both SAM and fibers will be referred to herein as “SAM/fiber webs.” It is common practice to prepare the SAM/fiber webs by first combining web components without the presence of water or other liquids (a practice that will be referred to herein as “dry mixing”) and laying out the mixture to form a web. While use of dry mixed SAM/fiber webs is prevalent, the fibers and SAM do not bond strongly to one another during dry mixing. As a result, there is a tendency for the SAM to migrate through the SAM/fiber web when the SAM/fiber web is moved, shaken, or otherwise agitated. This migration can result in an uneven distribution of SAM in the web, in which case the absorbent capacity of the material is not uniform throughout the absorbent material. It can also result in migration of SAM outside the web, leading to diminished absorbency and the potential for migration of SAM onto the user's skin or other body surfaces. Furthermore, because of the weakness of the connections formed by dry mixing, attempts to form SAM/fiber webs with very high concentrations of SAM fail because the components do not hold together. Since the SAM is the most important component of the web, increasing the maximum limits on web SAM content is desirable.

Efforts to reduce the potential for migration of SAM in SAM/fiber webs have focused on coating the SAM with fibers prior to combining the SAM with other fibers in a web. Several methods for coating SAM with fibers are known in the art. These methods have involved mixing the SAM and fiber under conditions that promote the formation of a bond between individual fibers and individual bodies of SAM, thereby coating the SAM with fiber. Examples of conditions that promote the formation of such bonds between fibers and SAM include, but are not limited to, combining the mixture with water, swelling the SAM with water prior to combining the fiber and SAM, application of a binder material to one or more components of the mixture, and the use of fibers that carry static electric charges. Individual particles, fibers, or other bodies of SAM that have been coated with fibers will be referred to herein as “coated SAM.” Several methods for coating SAM are known in the art. Examples include U.S. Pat. No. 3,901,236 to Assarson, et al., U.S. Pat. No. 5,436,066 to Chen, U.S. Pat. No. 5,489,469 to Kobayashi et al., U.S. Pat. No. 5,002,814 to Knack and Beckert, U.S. Pat. No. 5,800,418 to Ahr, and Europe Patent Publication No. EP 0478150A2 to Harada et al. Coating the SAM with fibers prior to placement in a web reduces migration of SAM because the fibers that coat the SAM can become entangled with fibers in the web and thereby help anchor the SAMs in place.

Although the use of coated SAMs has resulted in some reduction in SAM migration, additional improvements are desired. For example, the connection between fibers on adjacent bodies of coated SAM involves physical entanglement of fibers rather than the formation of any type of bond and thus does not prevent migration to the extent desired. Furthermore, existing process methods for coating SAM with fibers do not involve coating and formation of webs occurring simultaneously and therefore require manufacturers to incur the cost associated with including an additional step in the manufacturing process.

Use of coated fibers still places limits upon SAM concentration. First, using some fibers to coat the SAM and combining additional fibers (often referred to as carrier fibers) with the coated SAM to form the webs results in higher minimum fiber content requirements. Higher fiber content places limits on the concentration of SAM that can be placed in the web. Another limit arises from the fact that existing webs are generally held together by mechanical entangling of fibers. Increasing SAM content in the webs necessarily reduces the content of other web components, including fibers. With the existing webs, reductions in fiber content reduces the opportunities for mechanical entangling of the fibers. This loss of entangling means that the webs are held together less strongly and therefore have a higher susceptibility to migration of SAM. In fact, susceptibility of webs to migration in existing methods is a monotone nondecreasing function of SAM content. As used herein, the term “monotone nondecreasing function” means a mathematical function in which the dependent variable (in this case, susceptibility to SAM migration) of the function never decreases when the independent variable to which the function is applied, (in this case, relative SAM content), increases. In other words, if x≧y, then at all times f(x)≧f(y). Thus, a tradeoff exists in existing webs between the desire to maximize absorbency by increasing SAM content and the desire to avoid susceptibility to SAM migration resulting from decrease in fiber content. This tradeoff creates a disincentive for maximizing SAM content, which, in turn can place practical limitations on the amount of SAM content in a web.

What is needed in the art is a SAM/fiber web with very low susceptibility to SAM migration, even at very high concentrations of SAM. What is also needed is a method of making such webs. Webs having higher SAM content could significantly increase the efficiency of existing absorbent articles by allowing placement of greater absorbent capacity in a small area. In addition, it is possible that the diminished susceptibility of such a web to migration of SAM will remove the need for containing layers to hold absorbent structures in place. A method for making webs in which the coating and web formation can occur in a simultaneous process is also needed in the art.

### SUMMARY OF THE INVENTION

The present invention is directed to SAM/fiber webs that exhibit very low susceptibility to migration of SAM, even at very high concentrations of SAM. It has been found that a process involving first forming a SAM/fiber web using coated SAM in which the SAM has been swollen with a liquid, then subsequently removing the liquid absorbed in the SAM, results in SAM/fiber webs in which coated particles are strongly bonded to one another such that that migration of SAM in the webs is dramatically reduced. Because of the strength of the bonds in the present invention and the lack of need for any carrier fibers, it is possible to produce webs with significantly higher SAM concentration than achievable by existing processes. For the webs of the present invention, the susceptibility to SAM migration is not a monotone nondecreasing function of SAM content. In fact, in at least some embodiments the susceptibility of these webs to SAM migration is a monotone nonincreasing function of SAM content. As used herein, the term “monotone nonincreasing function” means a mathematical function in which the dependent variable (in this case, susceptibility to SAM migration) of the function never increases when the independent variable to which the function is applied, (in this case, relative SAM content), increases. In other words, if x≦y, then at all times f(x)≧f(y). In some embodiments the webs actually show less susceptibility to SAM migration when using a higher SAM content. While not wishing to be bound by any particular theory regarding the cause of this strong bonding and reduction in susceptibility to SAM migration, it is believed that the shrinking of the SAM that occurs upon removal of the absorbed liquid after SAM/fiber web formation causes wrinkles to form on the surface of the SAM. The wrinkles on a particle, fiber, or other body of SAM are believed to wrap around, clamp upon or otherwise entangle fibers that have coated other bodies of SAM that are located nearby in the SAM/fiber web. This wrapping or entangling effect is believed to cause the formation of physical bonds between adjacent particles or bodies of coated SAM and thereby to reduce susceptibility to SAM migration. It is also possible that swollen SAM forms bonds with the fiber attached to adjacent particles or bodies of coated SAM similar to the bonding that occurs between fibers and SAM when the SAM is coated. Another possibility is that the bonding occurs between the polymer contained in the SAM portion of adjacent particles or bodies of coated SAM.

The present invention is further directed to a method for making such webs. The webs of the present invention are formed from SAM that has been coated with fibers. The SAM is swollen with liquid at the time of web formation, and the liquid is subsequently removed. This method optionally allows embodiments in which the coating and web formation occur simultaneously. In one desirable embodiment, for example, the particles are coated by mixing swollen SAM and fibers and drawing the coated SAM directly into a chamber in which the web is formed while the SAM is still swollen. In another desirable embodiment, the coated SAM can be stored for some period between coating and web formation and any loss of SAM swelling during storage can be restored with additional liquid prior to web formation.

The present invention is further directed to absorbent articles comprising the SAM/fiber web of the present invention, and their applicability in disposable personal care products. The SAM/fiber webs of the present invention are particularly useful as absorbent components in personal care products such as diapers, feminine pads, panty liners, incontinence products, and training pants.

### BRIEF DESCRIPTION OF THE FIGURES

FIG. 1 illustrates a cross-sectional transverse view of a representative apparatus useful in the process of the present invention.

FIG. 2 illustrates a cross-sectional top view of a representative apparatus useful in the process of the present invention.

FIG. 3 illustrates a front elevation view of an apparatus that can be modified to be used in performing the Shakeout Test as discussed herein.

FIG. 4 illustrates a front elevation view of a part of the apparatus illustrated in FIG. 3 that can be modified to be used in performing the Shakeout Test as discussed herein.

FIG. 5 illustrates a front elevation view of an apparatus that can be used to perform the Shakeout Test as discussed herein.

FIG. 6 illustrates a side elevation view of an apparatus that can be used to perform the Shakeout Test as discussed herein.

### DETAILED DESCRIPTION OF THE INVENTION

The present invention is directed to SAM/fiber webs that possess low susceptibility to SAM migration even at high concentrations of SAMs. The present invention is further directed to a method for making such SAM/fiber webs. The present invention is further directed to absorbent articles containing the SAM/fiber web of the present invention.

The SAM/fiber web of the present invention contains multiple components. SAM is one component of the web. Examples of SAM include but are not limited to natural materials such as agar, pectin and guar gum, as well as synthetic materials such as synthetic hydrogel polymers. Examples of synthetic hydrogel polymers include, but are not limited to, carboxymethyl cellulose, alkali metal salts of polyacrylic acid, polyacrylamides, polyvinyl alcohol, ethylene maleic anhydride copolymers, polyvinyl ethers, hydroxypropyl cellulose, polyvinyl morpholinone, polymers and copolymers of vinyl sulfonic acid, polyacrylates, polyacrylamides, polyvinyl pyrridines, hydrolyzed acrylonitrile grafted starch, acrylic acid grafted starch, and isobutylene maleic anhydride copolymers and mixtures thereof. Partial crosslinking of hydrogel polymers will render the material insoluble in water but capable of swelling with water. The preceding list is not exclusive and persons skilled in the art will recognize that different SAMs or combinations thereof can be used. Persons skilled in the art will recognize that appropriate types and shapes or blends of types and shapes of SAM for use in the present invention will depend on the substances to be absorbed and the desired properties of the SAM/fiber web.

The SAM can be in any form suitable for use in absorbent composites including particles, fibers, flakes, spheres, and the like. Some SAM particles comprise two or more layers. The outermost layer or “shell” of such particles is comprised of one SAM while the innermost layer or “core” of the particle is comprised of a SAM that differs from the “shell” SAM with respect to monomer composition, degree of crosslinking, or other chemical or physical properties. Such particles are commonly referred to as “core-shell particles.” In one example of core-shell particles, the outer “shell” SAM serves to harden the particle because it has a greater degree of polymer crosslinking than the inner “core” SAM. Other SAM particles exhibit a gradient or gradual trend of increase or decrease of a chemical or physical property proceeding from the outer surface to the center of the particle. For example, some SAM particles have the highest degree of polymer crosslinking at the outer surface of the particle and a progressively lower degree of polymer crosslinking toward the center of the particle. Particles that exhibit such a gradual trend in crosslinking will be referred to herein as “gradient crosslinked particles.”

The fibers are another component of the SAM/fiber web of the present invention. Fibers used in the present invention can be a wide variety of natural or synthetic fibers, or a combination thereof. Persons skilled in the art will recognize that appropriate types or blends of types of fibers will depend on the substances to be absorbed and the desired properties of the SAM/fiber web.

Examples of natural fibers include, but are not limited to, woods, such as softwoods and hardwoods; straws and grasses, such as rice, esparto, wheat, rye, and sabai; canes and reeds, such as bagasse; bamboos; woody stalks, such as jute, flax, kenaf, and cannabis; bast, such as linen and ramie; leaves, such as abaca and sisal; and seeds, such as cotton and cotton linters. Softwoods and hardwoods are the more commonly used sources of cellulose fibers; the fibers can be obtained by any of the commonly used pulping processes, such as mechanical, chemimechanical, semichemical, and chemical processes.

Examples of softwoods include, by way of illustration only, longleaf pine, shortleaf pine, loblolly pine, slash pine, Southern pine, black spruce, white spruce, jack pine, balsam fir, douglas fir, western hemlock, redwood, and red cedar. Examples of hardwoods include, again by way of illustration only, aspen, birch, beech, oak, maple, eucalyptus, and gum. Softwood and hardwood Kraft pulps generally are desirable for toughness and tear strength, but other pulps, such as recycled fibers, sulfite pulp, and the like can be used, depending upon the application. Cellulose fibers can also be bleached to whiten the pulp fibers using various chemical processes.

Examples of synthetic fibers that can be used in the present invention include, by way of example and not by way of limitation, rayon, polyesters, polyamides, polyolefins, copolymers, and blends thereof. Examples of polyesters include, but are not limited to, polyethylene terephthalate, polybutylene terephthalate, polytetramethylene terephthalate, and polycyclohexylene-1,4-dimethylene terephthalate. Examples of polyamides include, but are not limited to, nylon 6, nylon 6/6, nylon 4/6, nylon 12, nylon 6/10, and nylon 12/12. Examples of polyolefins include, but are not limited to, polyethylenes (including without limitation high density polyethylene, medium density polyethylene, low density polyethylene, ultra low density polyethylene, linear low density polyethylene and others), polypropylenes (including, without limitation, isotactic polypropylene, atactic polypropylene, syndiotactic polypropylene, and others) and copolymers and blends thereof. The preceding list is not exclusive and persons skilled in the art will recognize that blends or copolymers of different polymers can be used. In addition, fibers that combine different polymer fibers in a multicomponent configuration can also be considered for use as a synthetic fiber.

An important consideration when selecting SAM/fiber web components is the relative sizes of the fibers and the SAM particles or fibers. This relationship can be expressed as a ratio of fiber length to diameter of a SAM particle or fiber. If this ratio is too low, the fiber will tend to be too short to allow interconnection of adjacent particles or bodies of coated SAM. If the ratio is too high, fibers will tend to be too long to coat the SAM in the coating step of the process. In one desirable embodiment involving SAM particles a ratio between about 1:1 and about 50:1 is employed. Another desirable embodiment involving SAM particles uses a ratio between about 2:1 and about 12:1. Another desirable embodiment involving SAM fibers uses a ratio between about 10:1 and about 120:1.

Persons skilled in the art will recognize that the SAM/fiber webs can contain other materials added to impart specific desired properties to the SAM/fiber web. Examples of additional components of the SAM/fiber webs include, but are not limited to, pigments such as titanium dioxide and iron oxide, odor absorbents such as zeolites and activated carbon, and dessicants such as silica gels and, molecular sieves.

Formation of the SAM/fiber web is also an element of the invention. Persons skilled in the art will recognize the variety of available methods that can be used to form webs. Webs can be made in a batch process in which formed webs are removed after each batch or through a continuous process. An example of a continuous process is one in which the web is laid out on a screen that is a moving conveyor that transports a continuous stream of formed web out of the web forming chamber. An example of one type of continuous web formation process is disclosed in U.S. Pat. No. 4,971,852 issued to Hammond. One desirable embodiment involves blowing a batch of coated SAM onto a stationary screen.

The SAM/fiber web of the present invention is formed while the SAM is swollen with liquid. In one embodiment, the SAM is swollen with liquid absorbed before or during the process of coating the SAM with fibers. Other possible embodiments include those in which the SAM is coated without swelling or in which the SAM loses the liquid with which it is swollen after coating. One way of making the SAM/fiber web of the present invention with these other embodiments would be to swell the SAM with liquid after coating and before web formation.

The appropriate degree to which the SAM is swollen, a value expressed as grams of liquid absorbed per grams of SAM, can depend on a number of factors. It is important that the SAM be sufficiently swollen at the time the web is formed to allow the formation of bonds between adjacent particles or bodies of coated SAM during subsequent removal of absorbed liquid. At least with embodiments that involve swelling SAM with distilled water, it has been observed that webs formed with SAM that is swollen to values greater than about 0.5 g/g at the time of web formation exhibit much less susceptibility to SAM migration than SAM/fiber webs formed with a lesser degree of SAM swelling. Many SAMs can be swollen to values of 200 g/g or higher. It has been observed, however, that SAMs tend to soften significantly when swollen close to their limit. Softening of the surface can cause SAM to lose shape. Persons skilled in the art will recognize embodiments in which it will be desirable to avoid this loss of shape caused by swelling the particles close to their limit.

Additional limits on the degree of swelling can be a factor with core-shell particles and gradient crosslinked particles, two forms of SAM discussed in greater detail above. It is desirable to avoid swelling the more highly crosslinked regions of the particles to a degree that will result in loss of integrity in the more highly crosslinked regions. For at least some types of core-shell particles and gradient crosslinked particles, a loss of shell integrity has been observed to occur above approximately 40 g/g.

Aside from the considerations noted in the preceding paragraph, the extent of swelling should be determined by balancing competing concerns. Higher degrees of swelling will result in a greater degree of SAM shrinking during removal of the absorbed liquid and thus more opportunity for the wrinkling that is believed to result in wrapping or entangling fibers attached to adjacent particles or bodies of coated SAM. On the other hand, adding more water will mean that more water will need to be removed. Additional water can require changes to the procedures for water removal (for example, if the water is evaporated, drying at a higher temperature and/or longer drying times) to shrink the SAM after web formation. Persons skilled in the art will recognize that selecting a specific degree of swelling will require consideration of the process specifics, the specific materials involved, and production goals of an individual embodiment.

The liquid used to swell the SAM should be readily absorbed by the SAM used and capable of achieving the desired degree of swelling noted above. One desirable embodiment uses distilled water to swell the SAM. Another desirable embodiment uses tap water.

The SAM of the present invention is coated with fibers. Coating is accomplished by a variety of mixing techniques that are well known in the art. These include, but are not limited to, blending, hand mixing, kneader-mixing, and placing the components together in an air suspension, as in an air agitation chamber or a fluidized bed. In one desirable embodiment, coating occurs after swelling the SAM by mixing the fibers and swollen SAM in the presence of air agitation. This embodiment is considered desirable because it reduces the tendency in some mechanical mixing techniques for particles or bodies of SAM to cluster together and thereby result in a web with less homogeneous SAM distribution.

After web formation, the water is then removed from the SAM to allow for the shrinking of SAM that is believed to result in formation of bonds between adjacent particles or bodies of coated SAM. One desirable embodiment involves allowing the water to evaporate. The embodiment can use either air drying at room temperature or accelerated drying at a higher temperature. Several heat drying methods are known to persons skilled in the art.

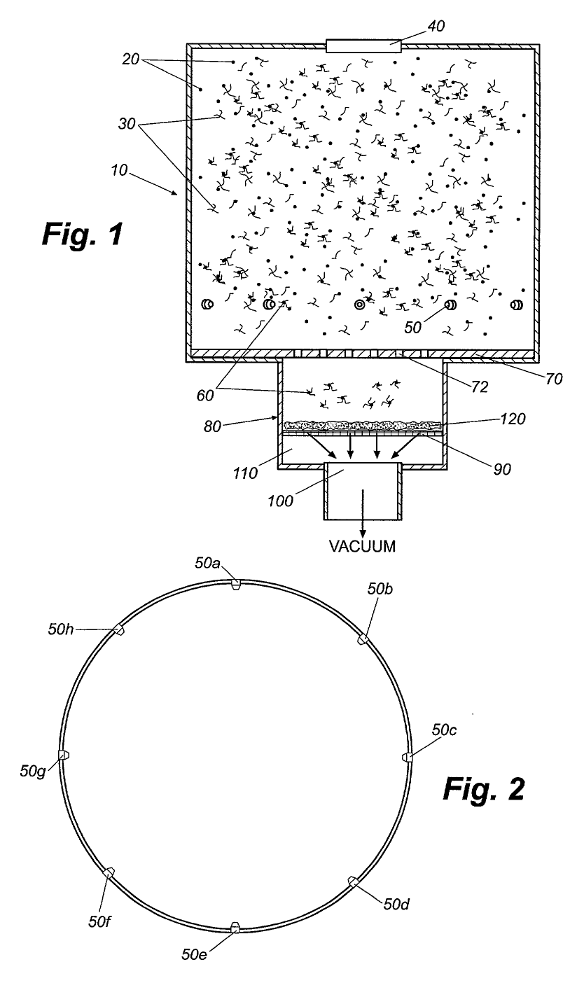

In one embodiment for making the webs of the present invention, SAM is coated with fibers by dry mixing fibers with swollen SAM. The SAM/fiber web is then formed of the coated SAMs while the composites are swollen with liquid. Finally, the SAM is permitted to dry to allow the swelling to recede. Referring to FIG. 1, in this embodiment the coating takes place in an air agitation chamber (10). Particles of SAM (20) are swollen with ten grams of distilled water per gram of SAM (g/g) prior to introduction into the chamber. The particles (20) are then introduced along with fibers (30) through an opening (40) in the air agitation chamber (10). Air agitation is provided with a number of air jets (50) uniformly spaced around the sides of the chamber and fired intermittently using the pulse created with a rotary pulse valve (not pictured). The fibers (30) then coat the swollen particles (20) to form coated particles (60). The coated particles (60) then pass through holes (72) in the floor (70) of the air agitation chamber (10) and into a web forming chamber (80) in which a screen (90) is located. An opening (100) located in a chamber (110) below the screen (90) pulls a vacuum so that negative pressure is maintained in both the air agitation chamber (10) and the web forming chamber (80). The vacuum serves to draw the coated particles (60) through the floor (70) of the air agitation chamber (10) and also serves to draw the coated particles (60) onto the screen (90) to form a web (120). The resulting web (120) is then completely dried in a convection dryer (not shown), air drying or a combination thereof. As used herein, “dried” refers to causing or allowing evaporation of all of the water absorbed in the SAM. The entire device, including both the air agitation chamber (10) and the web forming chamber (80) may be optionally enclosed in a plexiglass container (not pictured) such that the only openings in the container are the opening (40) in the air agitation chamber and the opening (100) that pulls the vacuum.

Residence time in the air agitation chamber (10) determines the extent to which the particles are coated. Persons skilled in the art can control residence time in the disclosed embodiment by varying the number and spacing of the nozzles (50), the air flow through the nozzles (50), the vacuum drawn on the two chambers through the duct opening (100), and the size and number of holes (72) in the floor (70) of the air agitation chamber (10). In general, residence time should be long enough to allow desired coating. It was observed that residence times less than 30 seconds generally did not allow sufficient coating of the SAM. Coating the SAM for too long will increase production time and can cause structural damage to the SAM, or can dry the SAM prior to formation of the SAM/fiber web. One desirable embodiment involved a residence time between about one and about five minutes. Another desirable embodiment involved a residence time of about three minutes.

Absorbent articles comprising the web of the present invention are another element of the present invention. Examples of absorbent articles include, but are not limited to, diapers, training pants, adult incontinence products, feminine care products, and absorbent wipes. Several methods for manufacturing absorbent articles using absorbent webs are well known in the art. Persons skilled in the art will recognize that such known methods can be used to manufacture absorbent articles using the web of the present invention.

The susceptibility of a SAM/fiber web to migration of SAM can be measured using a procedure referred to herein as the “Shakeout Test.” The Shakeout Test involves agitating web samples in a controlled fashion and determining the total loss of web mass from the sample. A sample of the SAM/fiber web is prepared in the shape of a circle with a diameter of three inches with a density of approximately 0.2 grams per cubic centimeter. The density of a sample can be adjusted to achieve this value by increasing or decreasing the thickness of the sample. Thickness can be decreased by pressing the sample without heat. Thickness can be increased by using a mechanical method to disrupt the web. One example of a method for mechanically disrupting a sample is repeatedly bending the sample by hand in several locations.

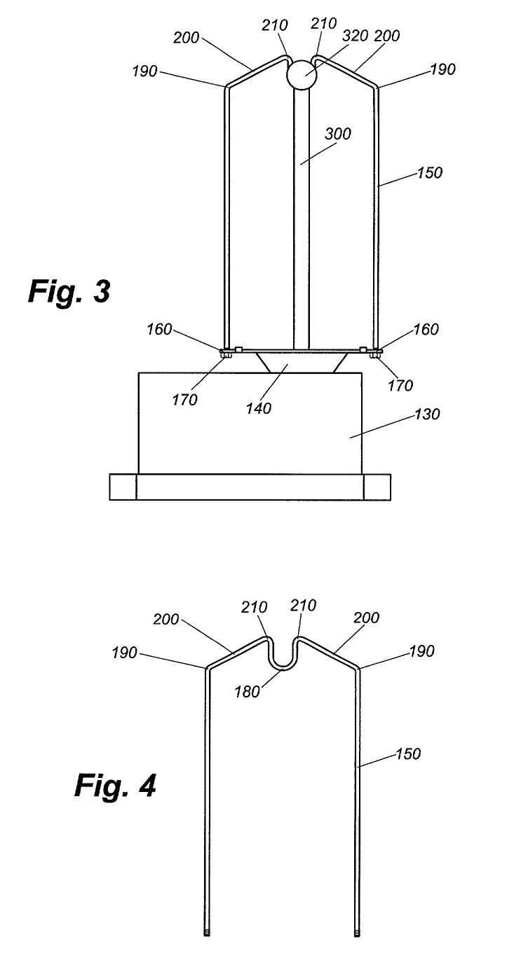

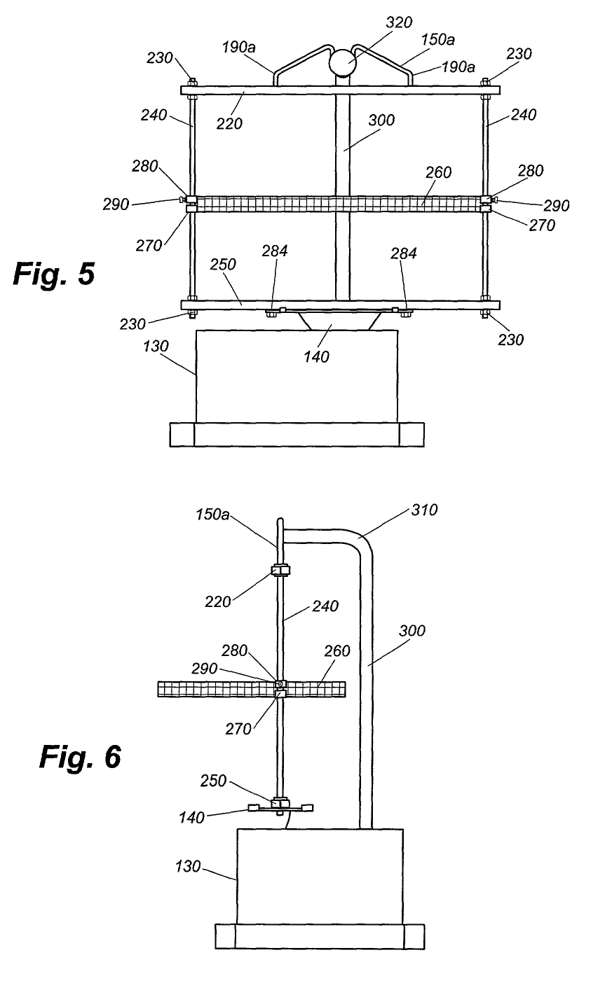

The Shakeout Test is conducted using the apparatus shown in FIG. 5 and FIG. 6. The apparatus was constructed by modifying a MODEL #RX-24 PORTABLE SIEVE SHAKER (herein after referred to as “RX-24”) available from W. S. Tyler Inc. FIG. 3 depicts an unmodified RX-24 and FIG. 4 depicts the guide frame portion of the apparatus depicted in FIG. 3 Referring now to FIG. 3 and FIG. 4 the shaking unit (130) of the RX-24 contains a motor that vibrates a sieve base (140). The RX-24 includes a guide frame (150) (Tyler Model No. R-445) comprised of a steel rod that is 0.3125 inches in diameter. As manufactured, the end prongs of the guide frame (150) are structured such that they can be inserted into two circular holes (160) on the sieve base (140) and fixed in place with nuts or screw caps (170). The holes (160) are cut through the sieve base (140) on a vertical axis. The two end prongs of the guide frame (150), as well as the holes (160) on the sieve base (140), are spaced nine and one quarter inch apart. The guide frame (150) as manufactured is 18.75 inches in height. At the opposite end from the threads on the prongs, the guide frame is configured into a series of bends that include a unshaped bend (180). In that series of bends, each prong has a 65 degree bend (190) into another length (200). That length (200) continues along a diagonal path for a horizontal distance of 3 and 7/16 inches into another bend of 115 degrees (210). The two prongs then form the unshaped bend (180), in which the prongs connect. The width of the inner dimension of the unshaped bend (180), which is a horizontal dimension in FIG. 4, is 1.0925 inches. The center line of the guide frame in the bottom of the unshaped bend (180) is 2 inches from the center line of the guide frame in the highest point in the nearest bends (210) in each prong.

Referring further to FIG. 3, the RX-24 also includes a ¾″ diameter metal rod (300) comprised of a stiff steel that attaches to the shaking unit (130). The ¾″ metal rod (300) is not directly shaken by the RX-24 but rather serves to provide stability. The ¾″ metal rod (300) runs vertically and has a 90° bend (310) (shown on FIG. 6) toward the front of the unit such that it runs horizontally after the bend (310). The rod (300) is of a length such that the center line of the horizontal length of the rod is 19 and one quarter inches above the surface of the shaking unit. (130). The rod (300) ends in a location directly above the sieve base (140). Attached to the end of the rod (300) is a guide (320), which is a plastic circular structure. The axis of the guide (320) is attached to the rod (300) and is 1¾ inch in diameter and five eights of an inch in width. The guide (320) has a shape similar to that of a narrow spool or pulley with a shallow groove with a depth of ⅜ of an inch running around the circumference of guide (320).

The motor in the RX-24 shakes the sieve base (140) approximately 7/16 inch vertically and approximately 11/32 inch horizontally. The horizontal movement is approximately parallel with the horizontal dimension of the upper length (200) of the guide frame (150). That is, the upper length (200) of the guide frame (150) moves in a vector that has both horizontal and vertical components, and the horizontal movement of the sieve base (140) is parallel to the horizontal component. The unshaped bend (180), (shown in FIG. 4) is placed in the groove of the guide (320), such that the guide (320) serves to limit the degree of motion for the guide frame (150) and thereby control the extent that the guide frame (150) can be agitated.

The Shakeout Test uses a modified RX-24 because it uses the RX-24 differently from the way in which sieve shakers are normally used. Sieve shakers such as the RX-24 are normally used to sort solids by size. One or more sieves containing screens are attached to the two vertical prongs of the guide frame (150) such that each screen is parallel to the floor and perpendicular to each vertical prong of the guide frame (150) is attached to one end of each screen. Multiple screens with different mesh sizes are attached when the solids are to be sorted into more than two size categories. Particulates are place in the sieve, (the top sieve when more than one sieve is used). The motor then shakes the sieve base (140) and solids are sorted based on whether they are small enough to pass through each screen.

For use in the Shakeout Tests, the RX-24 is modified to shake web samples and allow determination of the web's susceptibility to SAM migration based on the mass of web lost during shaking. The modifications involve making changes to the guide frame (150). Referring now to FIG. 5 and FIG. 6, the modified guide frame (150a) is cut so that the prongs have a length of only ⅞ inch below the 65 degree bend (190a). The cut end of the prongs are welded to an upper tie bar (220). The upper tie bar (220) is comprised of a stiff steel that is 22 inches in length, and is rectangular in cross section. As attached, the upper tie bar (220) has a horizontal thickness of 1 inch in the dimension that is perpendicular to the long axis of the bar and a vertical thickness of ½ inch. As welded in place, the modified guide frame (150a) is positioned over the center 9 and ¼ inches of the upper tie bar (220). The upper tie bar (220) includes two circular holes (not pictured) that run on a vertical axis, and that are centered on a point located ¾ inch from each end of the upper tie bar (220). Vertical rods (240) made of a stiff steel and having a diameter of 5/16 inch are inserted into the circular holes (not pictured) on the upper tie bar (220). The ends of the vertical rods (240) are threaded and the intersections of the upper tie bar (220) and the vertical rods (240) are secured by threading nuts or screw caps (230) onto the vertical rods (240) both below and above the intersection with the upper tie bar (220). A lower tie bar (250) of identical dimensions and composition to that of the upper tie bar (220) is attached to the opposite end of the vertical rods (240) in the same fashion that the upper tie bar (220) was attached. After the attachment of both tie bars (220, 250), the vertical distance between the two tie bars (220, 250), is 15 and ½ inches in length. A wire screen (260) is then attached to each of the vertical rods (240). The wire screen (260) is composed of 0.0625 inch diameter steel wire spaced approximately ½ inch apart to form square openings in the screen of about 7/16 of an inch in width. The wires in the wire screen (260) are connected to one another at the joints in a fashion that will not allow them to separate upon shaking or moving the wire screen (260). A vertical lip of wire screen approximately 0.9 inches in height runs around the edge of the wire screen (260). The wire screen (260) is attached to each vertical rod (240) at a location seven and ½ inches above the lower tie bar (250). Two stainless steel round bars (270, 280) with a 0.62 inch outer diameter, a 0.34 inch inner diameter, and 5/16 inch length are soldered to the wire screen (260) at each point of attachment such that the open ends of the round bar (270, 280) are on the upper and lower surface of the round bar (270, 280). The upper round bar (280) is positioned ¼ inch higher than the lower round bar (270). The wire screen (260) is fastened to the vertical rods (240) by running the vertical rods through the round bars (270, 280) and driving stainless steel screws (290) through a threaded hole that runs a radial path through the walls of the upper round bars (280) and firmly against the surface of the vertical rods (240) to hold the wire screen (260) in place. The wire screen (260) is oriented in a horizontal plane, is 20 inches long in the horizontal dimension parallel to the long axis of the tie bars (220, 250) and is 14 and ½ inches long in the horizontal dimension perpendicular to the long axis of the tie bars (220, 250). The wire screen (260) is fastened securely to the vertical rods (240) along the edge that runs in the horizontal dimension perpendicular to the long axis of the tie bars (220, 250), at a location that is 4½ inches from one corner of the wire screen (260). The lower tie bar (250) contains two circular holes (284) running along a vertical axis through the tie bar, the centers of which are at the endpoints of the central 9 and ¼ inches of the lower tie bar (250). The lower tie bar (250) is attached to the sieve base (140) by inserting two screws through the holes (284) and through the two holes (160), (not shown on FIG. 5 or FIG. 6; shown on FIG. 3) on the sieve base (140). Screws can be fixed in place by nuts or screw caps. Nuts and screw caps are selected and attached in a way that prevents any movement in the joints or attachments.

Other than the changes to the guide frame described in the previous paragraph, no additional components of the RX-24 are modified for purposes of performing the Shakeout Test. Numbers depicted upon FIG. 5 and FIG. 6 that are identical to numbers depicted in FIG. 3 refer to components of the RX-24 that are not modified.

As modified, the RX-24 is housed in a cabinet (not pictured) that does not affect the function of the unit but simply serves to provide sound insulation and other protection. Optionally, a tray, basin, or similar object (not pictured) can be placed at a location below the screen to catch any materials that fall from the screen during shaking. Any such tray, basin, or similar object used should be placed in a location such that it will not affect the motion of the parts that are shaken.

To perform the Shakeout Test using the modified RX-24, the web is laid horizontally flat (i.e. parallel to the floor) upon the wire screen (260) on the modified RX-24. The RX-24 then shakes the web at a frequency of approximately 520 cycles per minute for a period of two minutes. If any sheets of tissue paper have been placed above or below the web to facilitate lifting or handling, those sheets are removed prior to shaking.

After completion of the shaking portion of the test, mass loss is determined by comparing the total remaining mass of SAM/fiber web with the mass when placed on the screen. Specifically:

Mass loss (%)=100%×((M₀−M_{end})÷M₀)

Where:

M₀=web mass prior to test (grams)

M_{end}=web mass remaining after test (grams)

Lost mass of SAM/fiber web will generally fall through the openings in the screen. Any mass remaining on the screen is counted as web mass remaining.

While the foregoing discussion has described in detail one desirable method for conducting the Shakeout test using a specific type of apparatus, it will be appreciated that those skilled in the art will be able to prepare other apparatus that will allow equivalent testing in which agitation applied to webs will yield the identical results in terms of web loss as that achieved by the disclosed Shakeout Test. Accordingly, the scope of the Shakeout Test will include any equivalent test methods for determining web loss.

### EXAMPLES

### Example 1

A SAM/fiber web of three inches in diameter was prepared using the device in FIG. 1. Particles of cross-linked, partially neutralized sodium polyacrylate SAM with a diameter in a range of 300 to 600 microns were used to form the web. FAVOR® 880 particles available from Stockhausen, Inc. were used in this example. The particles were swollen with distilled water to 10 g/g prior to coating. Swelling in this example was accomplished by mixing the particles with the amount of water necessary to swell the particles to 10 g/g in a vessel and gently swirling the vessel containing the mixture by hand until the particles absorbed the entire volume of water. Once the particles had completely absorbed the volume of water, the swollen particles were immediately introduced into the air agitation chamber along with wood pulp fibers in which the pulp content was 16% hardwood and 84% softwood based on dry fiber weight. CR-1654 fibers available from Alliance Forest Products were used in this example.

In this example the air agitation chamber was comprised of stainless steel, and was six inches high with a cylindrical shape and a horizontal circular diameter of four and ¾ inches. There were a total of eight pulse jets spaced evenly around the circumference of the chamber at a height of ⅝ inch from the bottom of the chamber. Each air jet was fitted with nozzles having an opening diameter of 5/64 of an inch. Pressure of about 25 pounds per square inch was applied to the jets. The compressed air source for the jets was supplied through a rotary pulse valve (not pictured) configured such that the jets (50) would pulse in a specific sequence.

Referring now to FIG. 2, which is a horizontal cross-sectional view of the agitation chamber (10), the valve (not pictured) was configured such that jets (50a) and (50b) would pulse simultaneously, followed then by jets (50g) and (50h) pulsing simultaneously, followed then by jets (50c) and (50d) pulsing simultaneously, followed then by jets (50e) and (50f) pulsing simultaneously, followed then by a repeat of the same sequence beginning with a simultaneous pulse of jets (50a) and (50b). The rotary pulse valve (not pictured) was further configured such that each pair of jets pulsed about 20 times per minute. The floor of the chamber contained 69 round holes covering the circular space that occupied the center 2⅜ inches of the chamber floor. The holes were 7/32 of an inch in diameter and were arranged in a square row (i.e. grid) pattern spaced on centers that were 5/16 of an inch apart, leaving 3/32 of an inch of metal between adjacent holes in a row. The vacuum drawn was 1.5 inches of water measured at the opening (100), shown in FIG. 1. Using these parameters, a residence time of three minutes was achieved.

Coated particles drawn from the air agitation chamber were then laid out as a SAM/fiber web in a batch process. The web forming chamber was a cylindrical chamber one and ¼ inch in height and three inches in diameter and formed SAM/fiber webs shaped into disks also having a three inch diameter. The screen used at the bottom of the chamber was a U.S. Sieve Series 100 mesh. A combined mass of fibers and particles were used for such webs. The batch process was run until virtually all particles were drawn into a web. In the disclosed embodiment, this was completed after approximately three minutes after introduction of the SAM and fibers into the air agitation chamber.

For each sample, a sheet of tissue paper was placed over the screen in the web forming chamber prior to starting the process to facilitate lifting and handling of the sample after web formation. It was necessary to use a tissue paper that was sufficiently porous that it did not significantly impair flow of air through the screen. In this example, 9.8 pound White Forming Tissue available from American Tissue Inc. was used. “9.8 pound” denotes the fact that the tissue had a basis weight of 9.8 pounds per 2880 square feet.

Webs with a dry weight of 400 grams per square meter were prepared using a combined mass of SAM and fibers of 1.82 grams dry weight. At the time of introduction into the air agitation chamber, however, the SAM was swollen to contain 10 grams of water per gram of SAM. For example, a web containing 50% fibers and 50% SAM by weight would contain 0.91 grams each of SAM and fibers by dry weight. The SAM would contain 9.1 grams of water at the time of introduction into the agitation chamber, however, and would thus have a mass of 10.01 grams at that time.

After web formation was complete, a second sheet of tissue paper of the same type as the first sheet was placed over the resulting webs and the resulting webs were gently compressed by hand to facilitate lifting and handling. The webs were then completely dried, either by being placed in a convection dryer at a temperature of 60° C. for a period of at least three hours or by being placed in such a dryer at a temperature of 60° C. for a period of at least two hours and further allowing the samples to air dry for at least 24 hours. After drying and cooling, the webs were compressed without heat to achieve a density of about 0.2 grams per cubic centimeter. A Model #2833 Press manufactured by Fred S. Carver, Inc. was used to compress the webs, although those skilled in the art will recognize that a wide range of presses and similar devices can be used for this purpose. Successive compressions were used to attain the desired density. Density was determined by measuring the thickness of the web after each successive compression and using web mass and diameter to calculate density.

Webs were prepared with 60%, 70%, 80%, and 90% SAM content by dry weight and the remaining dry weight constituting fibers. Persons skilled in the art will recognize that webs made using other fibers, other shapes and compositions of SAM, and other percentages of SAM and fiber will work with this embodiment, and that the webs of the present invention that contain other components along with the SAM and fiber can be made with this embodiment.

### Comparative Example 1

For purposes of comparison, webs were formed using the detailed embodiment described in EXAMPLE 1, except that the SAM was not swollen with any liquid at any point during the process.

### Example 2

Shakeout Tests were conducted on samples made in EXAMPLE 1 having 60%, 70%, 80%, and 90% SAM content by dry weight and on samples made in Comparative Example 1 having 60%, 70%, 80%, and 90% SAM content by dry weight. Results are presented in Table 1.

TABLE 1
Web loss during Shakeout Test

| SAM Content | Example 2 | Comparative Example 1 |
|---|---|---|
| 60% | 3% | 26% |
| 70% | 4% | 57% |
| 80% | 1% | 82% |
| 90% | 1% | —* |

*Comparative Example 1 at 90% SAM content did not exhibit sufficient structural integrity to allow it to substantially retain its shape without the structural reinforcement provided by the screen, and therefore disintegrated when any attempt was made to move the web.

### Comparative Example 2

For purposes of comparison with webs formed using coated particles that are not swollen at the time of web formation, webs were formed using coated SAM that was dried prior to web formation. The coated SAM was first prepared using the procedures and detailed embodiment described in EXAMPLE 1, except that the web forming chamber (80) chamber was removed from the apparatus. This modification was performed upon an embodiment of FIG. 1 that was surrounded by a plexiglass container (not pictured) such that the only openings in the plexiglass container were the opening (40) in the air agitation chamber (10) and the opening (100) that pulled the vacuum. This configuration assured that the vacuum would draw air and coated particles (60) through the holes (72) in the floor (70) of the air agitation chamber (10). A sheet of 9.8 pound White Forming Tissue available from American Tissue Inc. was placed between the holes (72) and the opening (100) that pulled the vacuum to catch the coated particles as they dropped through the holes (72) and to prevent them from being drawn into the opening (100) that pulled the vacuum. The coated particles were then completely dried using the drying procedures in EXAMPLE 1.

Webs were then formed from the coated and dried particles. This was accomplished by reattaching the web forming chamber (80) to the air agitation chamber (10) and the opening (100) that pulled the vacuum as shown in FIG. 1. The coated and dried SAM particles were then inserted into the opening (40) in the air agitation chamber (10) with the pulse jets and vacuum operating as described in EXAMPLE 1. Except for the fact that coated and dried particles were inserted in the opening (40) instead of a mixture of swollen particles (20) and fibers (30), the procedures for the web formation step were identical to the procedures used to coat the particles and form the web in EXAMPLE 1.

Shakeout Tests were conducted on samples having 60%, 70%, 80%, and 90% SAM content by dry weight. Results are presented in Table 2.

### Example3

Webs were formed using the detailed embodiment described in EXAMPLE 1, except that the coated SAM was dried after coating, then swollen again prior to web formation. Particles were coated without being formed into a web using the coating procedure set forth for COMPARATIVE EXAMPLE NO. 2. Coated particles were then completely dried using the drying procedures in EXAMPLE 1.

After drying was complete, the particles were swollen again with distilled water. Swelling was accomplished by mixing the coated particles with the amount of water necessary to swell the particles to 10 g/g in a vessel and gently swirling the vessel containing the mixture by hand until the particles absorbed the entire volume of water.

Once the particles had completely absorbed the volume of water, the swollen and coated particles were then immediately introduced into the apparatus depicted in FIG. 1, now with the coating chamber (80) reattached to the air agitation chamber (10) and the opening (100) that pulled the vacuum as shown in FIG. 1. Except for the fact that particles that were already coated and swollen were inserted in the opening (40) instead of a mixture of swollen particles (20) and fibers (30), the procedures for the web formation step were identical to the procedures used to coat the particles and form the web in EXAMPLE 1.

Tests were conducted on samples having 60%, 70%, 80%, and 90% SAM content by dry weight. Results are presented in Table 2.

TABLE 2
Web loss during Shakeout Test

| SAM Content | Example 3 | Comparative Example 2 |
|---|---|---|
| 60% | 3% | 9% |
| 70% | 4% | 15% |
| 80% | 1% | 17% |
| 90% | 2% | 58% |

While the specification has been described in detail with respect to specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A method for making a web, comprising:
1) each of the following, in no particular order:
a) causing one or more superabsorbent material to absorb at least one liquid, and
b) coating the superabsorbent material with fibers,
2) forming a web comprising the coated superabsorbent material while the superabsorbent material contains an absorbed liquid, and
3) allowing at least some of the liquid absorbed in the superabsorbent material to be removed after formation of the web.

2. The method according to claim 1, wherein allowing at least some of the liquid absorbed in the superabsorbent material to be removed after formation of the web comprises allowing at least some of the liquid to evaporate.

3. The method according to claim 2, wherein allowing at least some of the liquid to evaporate comprises exposing the formed web to conditions that accelerate the evaporation of the liquid.

4. The method according to claim 3, wherein the conditions that accelerate the evaporation of the liquid comprise an elevated temperature.

5. The method according to claim 1, wherein the liquid is selected from solutions and mixtures that comprise water.

6. The method according to claim 1, wherein the liquid comprises distilled water.

7. The method according to claim 1, wherein the amount of absorbed liquid present in the superabsorbent material at the time of web formation is at least about 0.5 grams of the liquid per gram of superabsorbent material.

8. The method according to claim 1, wherein the fibers comprise wood pulp fibers.

9. The method according to claim 1, wherein coating the superabsorbent material with fibers comprises combining the fibers and superabsorbent material in the presence of air agitation.

10. The method according to claim 1, wherein forming the web comprises depositing the coated superabsorbent material onto a surface.

11. The method according to claim 1, wherein forming the web further comprises combining the coated superabsorbent material with one or more additional fibers, particles, materials or combinations thereof.

12. The method according to claim 1, wherein the superabsorbent material comprises particles.

13. The method according to claim 12, wherein at least some of the particles comprise an outer layer comprising at least one type of superabsorbent material and an inner core comprising at least one other type of superabsorbent material that differs from the superabsorbent material in the outer layer.

14. The method according to claim 12, wherein at least some of the particles are comprised of SAM that exhibits a gradual trend of decrease in crosslinking proceeding from the outer surface of the particle to the center of the particle.

15. A method for making webs comprising fibers and superabsorbent material wherein increasing the concentration of superabsorbent material in the webs does not result in webs that exhibit more web loss when subjected to the Shakeout Test.
